(19) United States
(12) Patent Application Publication
(10) Pub. No.: US 2008/0213381 A1
(43) Pub. Date: Sep. 4, 2008

(54) Oral Drug Delivery System

(75) Inventors: Nitin Bhalachandra Dharmadhikari, Mumbai (IN); Yashoraj Rupsinh Zala, Mumbai (IN)

Correspondence Address: SUGHRUE MION, PLLC, 2100 PENNSYLVANIA AVENUE, N.W., SUITE 800, WASHINGTON, DC, 20037, US

(73) Assignee: PHARMACEUTICAL INDUSTRIES LIMITED, ANDHERI (EAST), MUMBAI (IN)

(21) Appl. No.: 11/886,220

(22) PCT Filed: Mar. 14, 2006
(86) PCT No.: PCT/IN2006/000092

§ 371 Date: Sep. 13, 2007

(30) Foreign Application Priority Data
Mar. 14, 2005 (IN) 277/MUM/2005

(51) Int. Cl. A61K 9/50; A61P 25/24

(52) U.S. Cl. 424/497

(57) ABSTRACT

The present invention provides an oral drug delivery system comprising—
a. a core comprising an active ingredient composition comprising therapeutically effective amount of at least one active ingredient and a pharmaceutically acceptable excipient, and
b. a coating surrounding the core, said coating comprising a water-insoluble polymer and a pH-dependent polymer,
wherein the oral drug delivery system is in the form of a coated tablet and includes a feature such that after a predetermined delay the coating is reliably removed fully or partially from one or more of the tablet surfaces upon contact with intestinal fluids, further wherein the feature is that the core further comprises a composition selected from a swellable composition and a reactive composition located in the immediate vicinity of one or more preselected surfaces from which the coating is desired to be fully or partially removed, wherein the coating is not removed from at least one of the surfaces.

### FIELD OF THE INVENTION

The present invention relates to an oral drug delivery system comprising a coating that is reliably removed fully or partially from one or more preselected surfaces of the system upon contact of the system with intestinal fluids.

### BACKGROUND OF THE INVENTION

Oral administration of a drug provides a plasma level time profile of a drug or its active or inactive metabolite, which can be modulated by the design of the drug delivery system or dosage form.

Drug delivery systems releasing the drug slowly over longer duration have been traditionally used to improve therapy by
improving patient compliance to dosage regimens through the decrease in the number of doses the patient has to take in a day, by providing desired effective plasma levels for therapeutic efficacy over the duration of therapy for example throughout the day including at night when the patient is asleep;
decreasing peak plasma levels when they are associated with side effects;
reducing side effects in chronic therapy by reducing the fluctuation in plasma levels seen after multiple dosing of conventional rapid release systems;
when the drug has local action on the gastrointestinal mucosa, to spread the release spatially over the whole of the gastrointestinal mucosa as the drug delivery system is transported in the mucosa by the motility of the gastrointestinal tract.

Drug delivery systems are also designed to release the drug at specific site in the gastrointestinal tract by use of pH-dependent coatings that dissolve in the pH environment at the specific gastrointestinal site. There is a need for designing cores for such coated systems wherein the cores provide rapid release without substantial delay over the specific site or region, for example a release initiated over a period of 1 minute to 30 minutes after encountering the particular site or pH. There is also a need for designing cores for such site-specific coated systems wherein the cores are designed to provide controlled release over the specific region, for example from the colon to the rectum.

Our co-pending application PCT/IN 04/00192 describes novel oral drug delivery system that comprises a core with an active ingredient composition and a coating surrounding said core. The system is designed to include a design feature comprising a swellable composition adjacent to a preselected surface of the composition and optionally other design features. On imbibing water, the swellable composition swells and exerts pressure on the coating, particularly at the preselected surface, and only that coating from the preselected surface is removed. At the same time, the coating maintains its physical form and rigidity on other surfaces of the composition. In embodiments of our invention that are designed to provide controlled release, the system can be designed so that coating from one of the preselected surfaces is removed, and active ingredient release occurs from the exposed surface. In such embodiments, the exposed surface area thus remains constant as the release occurs and the active ingredient is released at a uniform or zero-order rate from the system. The system is advantageous over other prior arts described herein below.

U.S. Pat. No. 4,839,177 (the '177 patent) discloses a system for controlled-rate release of active substances consisting of (a) a deposit core of a defined geometric form, comprising a polymeric material having a high degree of swelling and a gellable polymeric material, and (b) a support platform consisting of a polymeric material insoluble in aqueous fluids applied to the deposit core such that it partially coats the deposit core. The trademark, Geomatrix®, refers to this system. Although the system provides a uniform rate of release, the disadvantage of this system is that the rigid support platform can crack or flake before the active substance is completely released. U.S. Pat. No. 5,422,123 is an improvement over the '177 patent in that the support platform consists of polymer substances, which are slowly soluble and/or gellable in aqueous fluids, and plasticizers, such that the support platform does not crack or flake before the drug is completely released from the deposit core. Although the patents disclose systems wherein surface area of release is reduced by covering two or more surfaces of the deposit core, in practice such systems are difficult to manufacture at an industrial scale—especially systems wherein two lateral surfaces and one planar surface are coated by the support platform. For example, in example 2 of the '177 patent, the barrier layers were applied by immersing the core in a polymeric solution as far as the edge of its upper base, such that two lateral sides and one planar surface of the core are coated. In example 3 of the '177 patent, the barrier layers were coated on the lateral sides of the core by spraying or brushing the polymeric solution onto the sides. These methods, although possible on small experimental scale, are not feasible and reproducible on an industrial scale. In US 20020090394 A1, the system of the '177 patent is further modified by including an additional feature of a pH-dependent polymer coating, such that the release does not occur in the stomach, but occurs after the system empties from the stomach. This system is also disadvantageous in that partial coating/barrier layer(s) below the pH-dependent polymer coating cannot be easily applied on a manufacturing scale, if at least three of the four tablet surfaces are to be coated to provide assured zero-order or uniform release. The partial coating/barrier layer(s) may be applied according to US 20020090394 only on one surface, but this results in the area not remaining constant if the matrix erodes. Particularly, the system could transform to a simple matrix exposed on all sides to the gastrointestinal fluids upon detachment of the partial coating/barrier layer. It has also been observed that the partial coating/barrier layer can detach itself from the deposit core upon handling and transport. Our PCT application PCT/IN 04/00192 has none of these disadvantages and is easy to manufacture.

U.S. Pat. No. 5,650,169 provides a pharmaceutical tablet capable of releasing the active ingredients contained therein at subsequent times, the tablet being prepared by a process wherein a three-layered tablet core comprising a first drug-containing layer, an intermediate barrier layer and a third drug-containing layer are covered with an impermeable polymeric film. The first layer presents a raised top, which is removed by abrasion so as to allow contact of the abraded first layer surface with the environment. The composition of the barrier layer is designed to modulate release from the third layer of the tablet. A major disadvantage of this system is that it requires removal of the raised top layer by abrasion to provide a means for release of the components of the system. This may not be feasible at an industrial scale. Further, if the abrasion is not uniform, the release of the active ingredients will be affected. Our system described in PCT/IN 04/00192 is advantageous, requires no complex process of abrasion and is easy to manufacture.

U.S. Pat. Nos. 6,720,005 and 6,733,784 relate to coated, platform-generating tablets. The tablet hydrates and expands upon swallowing such that the membrane covering the coating ruptures mostly around the belly-band surface of the tablet due to swelling of the core, thereby exposing the belly surface of the core tablet to hydrating and eroding liquids. A disadvantage of the system is that the coating is not reliably removed from the belly-band surface always but may rupture at a different weak point. Thus, the surface area of exposure may vary. Also, the systems show a lag time of release of half an hour or more. Many shapes of the core have been suggested in the invention but some of these may accentuate the problems encountered during tablet manufacture. Also, the belly-band surface, which is exposed after the coating ruptures, has the least surface area and other more preferred surfaces are not exposed. Our system described in PCT/IN 04/00192 is advantageous in that one can select any surface or surfaces, rather than only the coating around the belly-band surface to be removed from the tablet.

### OBJECTS OF THE INVENTION

It is an object of the present invention to provide an oral drug delivery system which comprises a core and a coating surrounding the core and including design features such that upon contact with an aqueous environment, the coating is removed from one or more preselected surfaces, not in the stomach as in our system described in PCT/IN 04/00192, but after the system is emptied from the stomach.

A still further object is to provide an oral drug delivery system where the coating is removed in any part of the gastrointestinal tract after the system empties from the stomach.

It is an object of the present invention to provide the aforesaid oral drug delivery system, which is designed to provide controlled release an active ingredient contained therein.

It is a further object to provide an oral drug delivery system that provides the aforesaid objectives and is also easy to manufacture.

### SUMMARY OF THE INVENTION

The present invention provides coated oral drug delivery systems using novel technology for removing coatings upon contact with intestinal fluids. Various embodiments are summarized below—
A. An oral drug delivery system comprising—
   a. a core comprising an active ingredient composition comprising therapeutically effective amount of at least one active ingredient and a pharmaceutically acceptable excipient, and
   b. a coating surrounding the core, said coating comprising a water-insoluble polymer and a pH-dependent polymer,
   wherein the oral drug delivery system is in the form of a coated tablet and includes a feature such that after a predetermined delay the coating is reliably removed fully or partially from one or more of the tablet surfaces upon contact with intestinal fluids, further wherein the feature is that the core further comprises a composition selected from a swellable composition and a reactive composition located in the immediate vicinity of one or more preselected surfaces from which the coating is desired to be fully or partially removed, wherein the coating is not removed from at least one of the surfaces.
B. An oral drug delivery system as described in A wherein a further feature is included in the coating.
C. An oral drug delivery system as described in B wherein the feature is that the coating on selected surface or surfaces of the tablet is selected from defective coatings and reactive coatings.
D. An oral drug delivery system as described in A, wherein the active ingredient is an antidepressant agent.
E. An oral drug delivery system as described in A, wherein the ratio of water-insoluble polymer to the pH-dependent polymer ranges from about 1:1 to about 10:1 by weight of the coating composition.
F. An oral drug delivery system as described in E, wherein the system is coated to a weight gain of about 8% to about 15% by weight of the core.
G. An oral drug delivery system as described in A, wherein the water-insoluble polymer is ethyl cellulose and the pH-dependent polymer is methacrylic acid copolymer.
H. An oral drug delivery system as described in A, wherein the active ingredient composition is present as one or more layers and the swellable composition is present as one or more layers.
I. An oral drug delivery system as described in H, wherein the active ingredient present in the different layers may be the same or different.
J. An oral drug delivery system as described in A, wherein the active ingredient composition is a controlled release composition comprising therapeutically effective amount of an active ingredient and a release controlling excipient.
K. An oral drug delivery system as described in J, wherein the release controlling excipient is selected from the group comprising high viscosity grades of hydroxypropyl methylcellulose (HPMC), polyethylene oxide homopolymers, hydroxypropyl cellulose (HPC) and mixtures thereof.
L. An oral drug delivery system as described in K, wherein the release controlling excipient is used in an amount ranging from about 10% to about 50% by weight of the active ingredient composition.
M. An oral drug delivery system as described in H, wherein the system further comprises a second active ingredient composition, the active ingredient composition being a rapid releasing composition, and the second active ingredient composition containing the same active ingredient as the active ingredient composition, the second active ingredient composition being a controlled release composition.
N. An oral drug delivery system as described in A, wherein the swellable composition comprises a swelling agent.
O. An oral drug delivery system as described in N, wherein the swelling agent is selected from the group comprising a swellable excipient, a gas generating agent and mixtures thereof.
P. An oral drug delivery system as described in A, wherein the swellable composition comprises wicking agents.
Q. An oral drug delivery system as described in A, wherein the swellable composition comprises osmogents.
R. An oral drug delivery system comprising—
   a. a core comprising an active ingredient composition comprising at least one active ingredient and a pharmaceutically acceptable excipient, and
   b. a coating surrounding the core, said coating comprising a water-insoluble polymer and a pH-dependent polymer,
   wherein the oral drug delivery system is in the form of a coated tablet and includes a feature such that after a predetermined delay the coating is reliably removed partially from one of the tablet surface upon contact with intestinal fluids, further wherein the feature is that the core further comprises a composition selected from a swellable composition and a reactive composition in the form of an in-lay tablet located in the immediate vicinity of the preselected surface from which the coating is desired to be partially removed.
S. An oral drug delivery system comprising—
   a. a core composition comprising (i) an active ingredient composition comprising therapeutically effective amount of at least one active ingredient and pharmaceutically acceptable excipients, (ii) a swellable composition located in the immediate vicinity of one or more preselected surfaces, comprising a swelling agent selected from a swellable excipient, gas generating agents and mixtures thereof, and optionally wicking agents and/or osmogents, and
   b. a coating surrounding the core, said coating comprising a water-insoluble polymer and a pH-dependent polymer,
   wherein the coating is impermeable to water and the active ingredient in gastric fluids, but is permeable to water in the intestinal fluids, whereby upon gastric emptying the system imbibes water and the swellable composition swells to remove the coating on the preselected surface(s).
T. An oral drug delivery system as claimed in claim 4, wherein the antidepressant agent is selected from the group comprising venlafaxine, paroxetine, fluvoxamine, sertraline, bupropion and their pharmaceutically acceptable salts.

### DETAILED DESCRIPTION OF THE INVENTION

The present invention provides an oral drug delivery system comprising—
a. a core comprising an active ingredient composition comprising therapeutically effective amount of at least one active ingredient and a pharmaceutically acceptable excipient, and
b. a coating surrounding the core, said coating comprising a water-insoluble polymer and a pH-dependent polymer,
   wherein the oral drug delivery system is in the form of a coated tablet and includes a design feature such that after a predetermined delay the coating is reliably removed fully or partially from one or more of the tablet surfaces upon contact with intestinal fluids, further wherein the design feature is that the core further comprises a composition selected from a swellable composition and a reactive composition located in the immediate vicinity of one or more preselected surfaces from which the coating is desired to be fully or partially removed, provided further that the coating is not removed from at least one of the surfaces.

Accordingly, the coating is partially removed from the system but may be fully or partially removed from one or more preselected surfaces. Hereafter, unless it is specified by use of specific wordings that the coating is removed “from a surface”, the use of the term ‘partial removal of coating’ will refer to partial removal from the system and may be full or partial removal from the surface. By the term “fully removed from the surface” it is meant the full area of the preselected surface is exposed by complete removal from that surface, whereas by the term “partially removed from the surface” it is meant that only a part of the surface area of the preselected surface is exposed by partial removal of the coating from that surface.

The term “reliably” as used herein means that the coating is removed from the preselected surfaces and is not removed from any other non-selected weak point in the coating. In contrast, the prior art system of PCT publication no. WO 02/080887 teaches a system where the opening of the system occurs reliably in time i.e. at a predetermined time but the surface that would be removed is unpredictable; in the present invention the term “reliably” refers to the removal of coating from any one or more of a preselected surface. Further in contrast, prior art system disclosed in U.S. Pat. No. 6,720,005 and U.S. Pat. No. 6,733,784 rupture “mostly” around the belly-band area only and do not allow preselection of any other surface.

The term “controlled release” as used herein means that the release of the active ingredient from the system is modified to occur at a slower rate than that from an immediate release product, such as a conventional tablet or capsule.

The term “after a predetermined delay” as used herein means that the release of the active ingredient is avoided in the stomach and is effected predominantly during passage of the system through the small intestine. Preferably, the oral drug delivery system of the present invention is designed such that the release of the active ingredient is predominantly one to three hours after oral administration.

The term tablet as used herein means a pharmaceutical composition comprising an active ingredient composition, which is formed into a rigid non-spherical unit that retains its geometric shape on handling, transport, coating and other operations.

The term “highly swellable excipient” as used herein means superdisintegrants known in the art that swell to a significant extent in water, as well as pharmaceutically acceptable excipients that have a degree of swelling comparable to superdisintegrants.

The term “moderately swellable excipient” as used herein includes pharmaceutically acceptable excipients that swell in water to an extent less than that of the superdisintegrants. Preferably, these are hydrogel polymers that are known in the art to have a swelling capacity more than other hydrogel polymers known in the art to imbibe water to form gels without significant swelling.

The term “pH-dependent polymer” as used herein means a polymer that is insoluble in acidic gastric fluids, but is soluble at a higher pH. It may be soluble at pH of fluids in specific parts of the intestine in order to activate the mechanism for reliable removal of the coating from the preselected surface at the specified location in the intestinal tract. For example, it may be soluble in the colonic pH in order to deliver the active ingredient to the colon.

The oral drug delivery system of the present invention is designed such that the coating is removed fully or partially from a preselected surface or surfaces upon contact with an aqueous environment and not removed from at least one of the surfaces. The partial removal of the coating may be affected by several means and the design features enabling the same may be features of the coating or the core, or both, operating cooperatively. For example, the system may be designed such that the coating is soluble or dissolved after it is emptied from the stomach (acidic environment) into the intestine having a relatively higher pH, from one surface of the system, but not dissolved from the other surfaces of the system, thus becoming partially removed from the system. Herein, the coating on the preselected surface can be rendered soluble by inclusion of a composition in the immediate vicinity of the preselected surface, which composition contains agents capable of rendering soluble the coating on the preselected surface. Alternatively, the oral drug delivery system may be designed such that the coating is a defective coating and is ruptured and removed fully or partially from one or more preselected surfaces of the system upon contact with intestinal fluids. As used herein the term “defective coating” refers to coatings that are susceptible to rupture due to a weakness. The defective coating on the preselected surface may be made by creating a weakness in the coating by mechanical, chemical or electrical means, or by radiation, or by designing a brittle coating, or a thin coating, or a brittle and thin coating on the preselected surface or surfaces. The defect may also be instantly created on the preselected surface by leaching of components of the coating upon contact with the aqueous environment. The defect may be in the form of an apparent fault such as an indent or a tear or a cut or an etching, which beginning from the outer surface of the coating may penetrate only partially through the coating. The core may be designed with a swellable or a reactive composition in the vicinity of the preselected surface. In preferred embodiments of the present invention, the core is swellable, and the coating is impermeable to the active ingredient. In the present invention, the coating surrounding the core is impermeable to water and the active ingredient in gastric fluids, but is permeable to intestinal fluids. Upon emptying from the acidic environment the pH-dependent polymer dissolves at a specific location in the intestine. The dissolution of the pH-dependent polymer renders the coating permeable to water and therefore, water permeates into the core. In one embodiment, the core comprises a swellable composition in the immediate vicinity of one or more preselected surfaces. The swellable composition comprises swelling agents that upon imbibing water from the external environment swell, causing the coating to be removed from the preselected surface(s). In another embodiment, the core comprises a reactive composition comprising ingredients in the immediate vicinity to dissolve or disintegrate or weaken the neighboring coating.

Active ingredients that may be used in the pharmaceutical composition of the present invention may be selected from the following, viz. alcohol abuse preparations, drugs used for Alzheimer's disease, anesthetics, acromegaly agents, analgesics, antiasthmatics, anticancer agents, anticoagulants and antithrombotic agents, anticonvulsants, antidiabetics antiemetics, antiglaucoma, antihistamines, anti-infective agents, antiparkinsons, antiplatelet agents, antirheumatic agents, antispasmodics and anticholinergic agents, antitussives, carbonic anhydrase inhibitors, cardiovascular agents, cholinesterase inhibitors, treatment of CNS disorders, CNS stimulants, contraceptives, cystic fibrosis management, dopamine receptor agonists, endometriosis management, erectile dysfunction therapy, fertility agents, gastrointestinal agents, immunomodulators and immunosuppressives, memory enhancers, migraine preparations, muscle relaxants, nucleoside analogues, osteoporosis management, parasympathomimetics, prostaglandins, psychotherapeutic agents, sedatives, hypnotics and tranquilizers, drugs used for skin ailments, steroids and hormones. Particularly, preferred active ingredients for use in the present invention are those that are useful as antidepressant agents.

Antidepressants are agents that are used to treat mental depression and manic-depressive disorders. There are several groups of antidepressants. The tricyclic and related antidepressants and the monoamine oxidase inhibitors (MAOIs) have been joined by the selective serotonin reuptake inhibitors (SSRIs) (typified by fluoxetine, fluvoxamine, paroxetine and sertraline), the reversible inhibitors of monoamine oxidase (RIMAs), and more recently by the serotonin and noradrenaline reuptake inhibitors (SNRIs) (typified by venlafaxine). In addition there is a range of other compounds not usually categorized into groups that play an important role in the treatment of the various depressive disorders; drugs such as bupropion. Amongst these, drugs such as fluoxetine, fluvoxamine, paroxetine, sertraline, venlafaxine and bupropion are prescribed more commonly and have achieved huge commercial success.

The most common disadvantage with immediate release or conventional dosage forms of antidepressant agents is side effects such as nausea and vomiting, which are peak related, and which lead to patient non-compliance. Hence, there is a need for a controlled release dosage form of antidepressant agents that provides the therapeutic blood plasma levels required for the treatment of depression and other related indications, over a period of 24 hours, while significantly reducing the side effects associated therewith. Controlled release once-daily formulations of antidepressant agents would therefore be expected to have an advantage in that peak levels would be lowered and therefore, the peak-related effects are lowered. It is also known that side effects caused by active agents, such as nausea and vomiting, can be avoided/reduced if the active agent release can be delayed such that release, and therefore, absorption from the gastric milieu is avoided.

Paroxetine is a selective serotonin reuptake inhibitor indicated in the treatment of depression, obsessive compulsive disorder, panic disorder, social anxiety disorder, generalized anxiety disorder and post-traumatic stress disorder. Paroxetine exerts its action by potentiation of serotonergic activity in the central nervous system resulting from inhibition of neuronal uptake of serotonin. It is a potent and highly selective inhibitor of neuronal serotonin reuptake. Paroxetine is commercially available as Paxil® conventional immediate release tablets, and as Paxil® CR controlled release tablets.

Venlafaxine is a potent inhibitor of neuronal serotonin and norepinephrine reuptake, and is indicated for the treatment of major depressive disorder. Venlafaxine is commercially available in the United States of America as immediate release tablets under the brand name Effexor®, and as extended release preparation under the brand name Effexor® XR. The initial starting dose of venlafaxine is 75 mg/day, administered in two or three divided doses. Depending on tolerability and the need for further clinical effect, the dose may be increased to 150 mg/day. If there is a further need, it may be increased to 225 mg/day. When increasing the dose, increments of up to 75 mg/day are made at intervals of four days or more. Thus, a gradual upward dosage titration period of 2 weeks or more may be required to reach the optimum dosing regimen, in order to avoid discontinuation due to treatment emergent side effects such as nausea and vomiting. The use of conventional immediate release dosage forms of venlafaxine hydrochloride cause an immediate release of the drug upon oral administration, thereby leading to a rapid increase in plasma levels of venlafaxine and its active metabolite, with peak plasma concentrations being achieved at 2 hours and 4 hours, respectively. The elimination half-life of venlafaxine and its active metabolite is 5 hours and 11 hours, respectively. This leads to a sub-therapeutic plasma venlafaxine level at around 12 hours after oral administration of the conventional immediate release dosage form, thus requiring the administration of an additional dose.

U.S. Pat. No. 6,440,457 claims a method for administering a drug to the gastrointestinal tract of a human comprising venlafaxine in a controlled or sustained release dosage form, wherein the dosage form provides the drug over an extended period of time in a therapeutically responsive dose to produce antidepressant therapy.

U.S. Pat. No. 6,274,171 claims an extended release formulation of venlafaxine hydrochloride comprising a pharmaceutically acceptable capsule containing spheroids comprised of from about 6% to about 40% venlafaxine hydrochloride by weight, about 50% to about 94% microcrystalline cellulose, NF, by weight, and optionally from about 0.25% to about 1% by weight of hydroxypropyl methylcellulose, USP, wherein the spheroids are coated with a film coating composition comprised of ethyl cellulose and hydroxypropyl methylcellulose. This patent relates to the commercially available controlled release formulations of venlafaxine commercially marketed under the tradename Effexor®.

U.S. Pat. Nos. 6,403,120 and 6,419,958 relate to methods for providing therapeutic blood plasma concentration of venlafaxine over a twenty-four hour period with diminished incidence of nausea and emesis, which comprises administering orally to a patient in need thereof. However, in reported clinical studies, data does not indicate a significant benefit in reduction in incidence of nausea and vomiting. We have found that the system provides a high peak plasma level of venlafaxine at about 6-7 hours. There is therefore still a need to provide a controlled drug delivery system that provides plasma levels of venlafaxine over 24 hours that are therapeutically effective, and significantly reduce the treatment-emergent nausea and vomiting.

Embodiments of the present invention provide an oral drug delivery system comprising—
a. a core comprising an active ingredient composition comprising therapeutically effective amount of at least one active ingredient and pharmaceutically acceptable excipients, and
b. a coating surrounding the core, said coating comprising a water-insoluble polymer and a pH-dependent polymer,
   wherein the oral drug delivery system has at least two surfaces and is designed in a manner such that after a predetermined delay, the coating is removed fully or partially from one of the surfaces when the oral drug delivery system contacts intestinal fluids, and further wherein the coating is removed from the surface different from the one having the least surface area. The oral drug delivery systems of the present invention provide benefit over prior art systems which do not provide a flexibility in choice of the surface from which the coating can be removed, and generally expose the surface with the lower surface area.

Another embodiment of the present invention provides an oral drug delivery system comprising —
a. a core composition comprising (i) an active ingredient composition comprising therapeutically effective amount of at least one active ingredient and pharmaceutically acceptable excipients, (ii) a swellable composition located in the immediate vicinity of one or more preselected surfaces, comprising a swelling agent selected from a swellable excipient, gas generating agents and mixtures thereof, and optionally wicking agents and/or osmogents, and
b. a coating surrounding the core, said coating comprising a water-insoluble polymer and a pH-dependent polymer,
   wherein the coating is impermeable to water and the active ingredient in gastric fluids, but is permeable to water in the intestinal fluids, whereby upon gastric emptying the system imbibes water and the swellable composition swells to remove the coating on the preselected surface(s).

In one embodiment of the present invention, the active ingredient composition is a swellable composition comprising therapeutically effective amount of at least one active ingredient and a swelling agent. In another embodiment of the present invention, the core comprises active ingredient composition and swellable composition, which may be present as one or more layers. The active ingredient present in these layers may be the same or different.

The swellable composition used in oral drug delivery system of the present invention comprises a swellable agent that may be selected from a group comprising a swellable excipient, a gas generating agent and mixtures thereof. The swelling agent is generally used in an amount ranging from about 0.5% to about 99.9% by weight of the swellable composition, preferably from about 80% to about 99% by weight of the swellable composition. The swellable excipient that may be used may be a highly swellable excipient selected from vinylpyrrolidone polymers such as crospovidone; cellulose and cellulose derivatives such as crosslinked carboxyalkylcelluloses and their alkali salts; starch and starch derivatives, such as sodium starch glycolate, resins and mixtures thereof. The highly swellable excipient is preferably used in an amount ranging from about 2% to about 35% by weight of the swellable composition. The swellable excipient that may be used may be a moderately swellable excipient and may be used in an amount ranging from about 5% to about 70% by weight of the swellable composition, preferably about 50% to about 70% by weight of the swellable composition. Gas generating agents that may be used in the present invention include carbonates such as calcium carbonate, bicarbonates such as sodium or potassium bicarbonate, sulfites such as sodium sulfite, sodium bisulfite, or sodium metabisulfite, and the like. These salts may be used alone or in combination with an acid source as a gas generating couple. The acid source may be an edible organic acid, a salt of an edible organic acid, acidic components such as acrylate polymers, or mixtures thereof. Examples of organic acids that may be used include citric acid, malic acid, succinic acid, tartaric acid, fumaric acid, maleic acid, ascorbic acid, glutamic acid, and their salts, and mixtures thereof.

The swellable composition may further comprise a wicking agent in an amount ranging from about 0.5% to about 90% by weight of the swellable composition. Examples of wicking agents that may be used include, but are not limited to, silicified microcrystalline cellulose, colloidal silicon dioxide, kaolin, titanium dioxide, fumed silicon dioxide, alumina, niacinamide, sodium lauryl sulfate, low molecular weight polyvinylpyrrolidone, m-pyrol, bentonite, magnesium aluminum silicate, polyester, polyethylene. Preferably, the wicking agents used in the pharmaceutical composition of the present invention include cellulose and cellulose derivatives such as silicified microcrystalline cellulose, colloidal silicon dioxide, and mixtures thereof. In one embodiment of the present invention, the wicking agent used in a mixture of silicified microcrystalline cellulose and colloidal silicon dioxide, preferably in an amount of about 95% by weight of the swellable composition. The silicified microcrystalline cellulose that is preferred is commercially available under the tradename Prosolv®, having 2% w/w colloidal silicon dioxide and typical particle size in the range of 20-200 μm. Preferably, Prosolv® SMCC 90 having a particle size of 90 μm is used in an amount of about 80% to about 90% by weight of the swellable composition.

The swellable composition may also comprise osmogents in an amount ranging from about 0.5% to about 10% by weight of the swellable composition. Examples of osmogents that may be used include, but are not limited to, inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, raffinose; water-soluble amino acids such as glycine, leucine, alanine, or methionine; urea and the like; osmopolymers selected from the group consisting of poly(hydroxyalkyl methacrylate) having a molecular weight of 20,000 to 5,000,000; poly (vinylpyrrolidone) having a molecular weight of about 10,000 to 360,000; poly(vinyl alcohol) having a low acetate content and lightly crosslinked with glyoxal, formaldehyde, glutaraldehyde and having a degree of polymerization from 2,000 to 30,000; poly(ethylene oxide) having a molecular weight from 10,000 to 7,8000,000; acidic carboxy polymers known as carboxypolymethylene or as carboxyvinyl polymers, a polymer consisting of acrylic acid lightly cross-linked with polyallylsucrose and sold under the trademark Carbopol®, acidic carboxy polymer having a molecular weight of 200,000 to 6,000,000, including sodium acidic carboxyvinyl hydrogel and potassium acidic carboxyvinyl hydrogel; Cyanamer® polyacrylamide; and the like, and mixtures thereof.

In one embodiment of the present invention, the partial removal of the coating may be affected by including in the swellable composition gas generating agents as the swelling agents. The core of such an embodiment comprises the active ingredient composition and a swellable composition comprising the gas generating agents. The core is coated with a coating composition comprising a water-insoluble polymer and a pH-dependent polymer. A predetermined delay in release of the active ingredient contained in the core of the system is achieved due to presence of the pH-dependent polymer in the coating, which polymer dissolves only after the system empties from the stomach. The alkaline environment of the intestine causes the pH-dependent polymer to dissolve rendering the coating permeable to water. Thus, the fluid from the external environmental enters the system, and the gas generating agents present in the core release gas, generating pressure that causes the coating to be removed partially from a preselected surface. The preselected surface is that surface of the system that bears the swellable composition in its immediate vicinity. Preferably, the swellable composition may comprise a mixture of swellable excipients and gas generating agents, the mixture being obtained using types and amounts of components selected as described herein below and in examples that follow.

The active ingredient composition of the oral drug delivery system of the present invention may be designed to provide controlled release of the active ingredient contained therein. The release-controlling excipients that may be used in the active ingredient composition of the present invention to provide the controlled release of the active ingredient may be selected from hydrophilic polymers such as methyl cellulose, hydroxypropyl methylcellulose, hydroxyethyl cellulose, hydroxypropyl cellulose, hydroxyethyl methylcellulose, carboxymethylcellulose and sodium carboxymethylcellulose; hydrophobic compounds such as ethyl cellulose, glycerol palmitostearate, beeswax, glycowax, castor wax, carnauba wax, glycerol monostearate, stearyl alcohol, glycerol behenic acid ester, cetyl alcohol, natural and synthetic glycerides, waxes, fatty acids, hydrophobic polyacrylamide derivatives, hydrophobic methacrylic acid derivatives; vinyl pyrrolidone polymers such as polyvinylpyrrolidone and copolymers of vinyl pyrrolidone and vinyl acetate; alkylene oxide homopolymers; gums of plant, animal, mineral or synthetic origin; and mixtures thereof. The active ingredient composition may comprise one or more of the release controlling excipients mentioned above in an amount ranging from about 2% to about 90% by weight of the core. In preferred embodiments of the present invention, the active ingredient is an antidepressant agent, preferably venlafaxine or paroxetine, and the release controlling excipient is selected from high viscosity grades of hydroxypropyl methylcellulose (HPMC), polyethylene oxide homopolymers, hydroxypropyl cellulose (HPC) and mixtures thereof. Preferably, the release controlling excipient is HPMC, wherein 2% w/v solution of the HPMC has a viscosity in the range of 3000 mPaS to 120,000 mPaS. The high viscosity release controlling excipient is preferably used in an amount ranging from about 10% to about 50% by weight of the active ingredient composition, more preferably from about 20% to about 30% by weight of the active ingredient composition.

The coating used in the oral drug delivery system of the present invention is designed to obtain the desired release profile of the active ingredient, while providing a predetermined delay in release of the active ingredient. Thus, the coating comprises a water-insoluble polymer, a pH-dependent polymer and other pharmaceutically acceptable excipients. The pH-dependent polymer dissolves in the alkaline conditions of the intestine, i.e. when the oral drug delivery system is emptied from the stomach. The system does not release any active ingredient while it is passing through the stomach. Upon reaching the intestine, the pH-dependent polymer dissolves so that fluid from the external environment enters the system. The swellable composition then imbibes the fluid and swells to exert pressure on the coating, causing it to be removed on the side that has the swellable composition. Thus, a defined and controlled area is made available for release of the active ingredient.

The water-insoluble agents that may be used in the coating composition of the present invention to provide an active ingredient impermeable coating include, but are not limited to, cellulose derivatives such as cellulose acetate, ethyl cellulose and the like, pH-independent acrylates such as Eudragit RS, Eudragit RL and the like. Examples of pH-dependent polymers that may be used in the coating composition of the present invention include, but are not limited to, polymethacrylates such as poly(methacrylic acid, methylmethacrylate) 1:1, poly(methacrylic acid, ethylacrylate) 1:1, poly(methacrylic acid, methylmethacrylate) 1:2, and the like.

In one preferred embodiment of the present invention, a mixture of a water-insoluble polymer and a pH-dependent polymer is used to obtain the coating. For example, a mixture of ethyl cellulose and Acryl-EZE® (methacrylic acid copolymer type C), may be used to obtain the coating. The oral drug delivery system may be coated to a weight gain in the range of about 2% to about 15% by weight of the core. The pH-dependent polymer is used in an amount such that the active ingredient is released after a predetermined delay, i.e. after the system is emptied from the stomach. The ratio of the water-insoluble polymer to the pH-dependent polymer in the coating composition ranges from about 1:1 to about 10:1, depending upon the nature of the active ingredient used and depending upon the release profile desired. The amount of coating deposited on the core will also depend on this ratio.

In one embodiment of the present invention, the swellable composition is present as an in-lay tablet in a core comprising the active ingredient composition. The core with the in-lay tablet is coated with a coating comprising a water-insoluble polymer and a pH-dependent polymer. When the system is emptied from the stomach, the pH-dependent polymer dissolves and thereby allows water to enter the system. The swellable composition then swells to exert pressure and removes the coat, thereby exposing a surface area equivalent to the surface area of the in-lay portion. The active ingredient is then released from the exposed surface. Alternatively, the in-lay tablet comprises a reactive composition that interacts with the water-insoluble polymer in the coating in its immediate vicinity to dissolve or erode the same. For example, the water-insoluble polymer may be ethylcellulose containing not less than 46.5% ethoxy groups and the reactive composition may be a composition that liberates excipients that dissolve or erode the ethylcellulose. These compositions, for example, may comprise ethanol microencapsulated in a suitable water-soluble polymer, which dissolves upon contact with water to release the ethanol.

In another embodiment of the present invention, the core is compressed into a bilayer tablet; the first layer comprising the active ingredient composition having uninterrupted plain surfaces surrounded by coating or the second layer; and the second layer comprising the swellable composition having at least one surface interrupted by at least one depression or cavity. The pH-dependent polymer present in the coating dissolves upon contacting the intestinal fluids, and the water entering the system causes the coating to be removed from the surface having the depression or cavity.

The oral drug delivery system of the present invention may be obtained by processes conventional to the pharmaceutical art. The core may be obtained in the form of—(i) a single layer compressed tablet comprising the active ingredient composition and the swellable composition (or the reactive composition), (ii) a tablet comprising laminar layers of the active ingredient composition and the swellable composition (or the reactive composition), or (iii) an in-lay tablet wherein the active ingredient composition is compressed with the swellable composition (or the reactive core composition) in a manner such that the swellable composition forms an in-lay. The active ingredient composition and the swellable composition (or the reactive composition) may be obtained by processes such as wet granulation or dry granulation (typified by slugging, roller compaction), using conventional techniques. These two compositions may then be mixed and compressed to obtain a single-layered core, or may be compressed to obtain a layered tablet with the two compositions forming the two layers, or may be compressed to form an in-lay tablet. Compression is carried out by techniques known to a person skilled in the pharmaceutical art. Coating of the core with the suitable coating is then carried out by conventional processes. The coating composition may be obtained by dissolving or suspending the components in a suitable solvent system.

The examples that follow do not limit the scope of the invention and are merely used as illustrations.

### EXAMPLE 1

Oral drug delivery system comprising paroxetine hydrochloride was obtained as per the present invention, as detailed in Table 1 below.

TABLE 1

| | Quantity | |
|---|---|---|
| Ingredients | mg/tablet | % w/w |

First layer

| | | |
|---|---|---|
| Paroxetine hydrochloride hemihydrate | 42.66 | 24.38 |
| (equivalent to Paroxetine base 37.5 mg) | | |
| Hydroxypropyl methylcellulose | 40.00 | 22.86 |
| (HPMC, Methocel K100LV) | | |
| Polyvinylpyrrolidone (Povidone K-30) | 10.00 | 5.71 |
| Lactose monohydrate | 52.31 | 29.91 |
| Silicified microcrystalline cellulose | 27.00 | 15.43 |
| (Prosolv SMCC) | | |
| Colloidal silicon dioxide | 1.00 | 0.57 |
| Magnesium stearate | 2.00 | 1.14 |

Second layer

| | | |
|---|---|---|
| Silicified microcrystalline cellulose | 84.8 | 84.8 |
| (Prosolv SMCC) | | |
| Crospovidone | 10.0 | 10.0 |
| Colloidal silicon dioxide | 2.5 | 2.5 |
| Sodium lauryl sulfate | 1.0 | 1.0 |
| Color (FD&C blue lake no 1) | 0.4 | 0.4 |
| Magnesium stearate | 1.05 | 1.05 |
| Talc | 0.25 | 0.25 |

Coating

| | | |
|---|---|---|
| Aquacoat ECD 30 solids (aqueous | 21.34 | Coated to a weight |
| ethyl cellulose dispersion) | | gain of about 12% |
| Acryl eze white 9318509 | 11.75 | by weight of the |
| Dibutyl sebacate | 1.60 | bilayered core |
| Triethyl citrate | 0.64 | |

Paroxetine hydrochloride hemihydrate, HPMC, lactose monohydrate and povidone K-30 were passed through ASTM (American Society for Testing and Materials) sieve #40 and mixed suitably. The mixture thus obtained was granulated with purified water to a suitable end-point, and the granules obtained were dried to a moisture content of about 1-2%. The dried granules were milled suitably and lubricated with a mixture of Prosolv SMCC 90, colloidal silicon dioxide and magnesium stearate, to obtain the blend for the first layer.

Silicified microcrystalline cellulose, crospovidone, sodium lauryl sulfate and a suitable color were passed through ASTM sieve #40 and mixed suitably. The blend so obtained was lubricated with a mixture of colloidal silicon dioxide, talc and magnesium stearate (previously passed through ASTM sieve #60).

The above two preparations were compressed to obtain bilayer tablets, which were coated with an aqueous dispersion containing ethyl cellulose, Acryl-Eze, dibutyl sebacate and triethyl citrate to a weight gain of about 12% by weight of the core.

The tablets thus obtained were subjected to dissolution testing using United States Pharmacopoeia dissolution apparatus, type I, using 900 ml of pH 6.8 phosphate buffer as the dissolution medium, at 100 rpm. The results of the dissolution test are recorded in Table 2 below.

TABLE 2

| Time (hours) | Percent drug released |
|---|---|
| 1 | 5 |
| 2 | 13 |
| 4 | 31 |
| 6 | 56 |
| 8 | 83 |
| 10 | 94 |
| 12 | 94 |

The release from the controlled release layer of the system was zero-order, i.e. linear over time (regression co-efficient r² being 0.9895).

The composition has a core in the form of a bilayered tablet with a colored layer comprising the swellable composition, a colorless layer comprising the active ingredient composition, and a colorless coating surrounding the core. Thus, the finished product clearly shows the presence or absence of the active ingredient composition—i.e. even if the two layers in the core were to separate during processing or other operations, the absence of any of the layers would be visible to the naked eye and such a tablet would be easy to discard. This is a clear advantage over prior art products, such as those obtained by the '177 patent, wherein separation or loss of any of the layers would not be known and there is a risk of the patient consuming only the barrier layer composition (no active ingredient would be administered at all in such a case), or only the deposit core composition (absence of the barrier layer would affect the release profile of the active ingredient and thereby affect the patient.)

### EXAMPLE 2

Paroxetine controlled release compositions were prepared similar to example 1, except that the coating amount was varied to study the effect of coating percent on release of paroxetine or its pharmaceutically acceptable salt. Bilayered cores similar to those in example 1 were coated with a coating composition similar to that in example 1, but were coated to a different weight gain. The tablets so obtained were subjected to dissolution test to determine the time required for the coating to rupture on the side of the second layer, i.e. the swellable composition.

The tablets were initially placed in 0.1N hydrochloric acid for 2 hours. None of the tablets ruptured or opened during this time. This indicates that the coating has sufficient acid resistance and would provide release of the paroxetine hydrochloride only after reaching the intestine, where the pH is alkaline.

The tablets were then placed in pH 6.8 phosphate buffer and observed for rupture or opening, i.e. the time required for the coating to rupture on the side of the swellable layer and provide a defined surface area for release of the active ingredient. The results are recorded in Table 3 below.

TABLE 3

| Weight gain upon coating | Opening time |
|---|---|
| About 8% by weight of core | 7-9 minutes |
| About 10% by weight of core | 11-19 minutes |
| About 12% by weight of core | 21-28 minutes |

The coating composition can thus be coated to different weight gain to delay release of the active ingredient.

### EXAMPLE 3

An oral controlled drug delivery system of venlafaxine was obtained as per Table 4 below.

TABLE 4

| | |
|---|---|
| Ingredients | Quantity (mg/tablet) |

First layer

| | |
|---|---|
| Venlafaxine hydrochloride (equivalent to | 42.44 |
| 37.5 mg of venlafaxine base) | |
| Hydroxypropyl methylcellulose (HPMC K4M) | 12.00 |
| Polyvinylpyrrolidone (PVP K-30) | 10.00 |
| Lactose monohydrate impalpable | 30.06 |
| Eudragit L-100/55 | 20.00 |
| Magnesium stearate | 0.75 |
| Talc | 0.75 |

Second layer

| | |
|---|---|
| Silicified microcrystalline cellulose (Prosolv | 84.80 |
| SMCC 90) | |
| Crospovidone | 10.00 |
| Colloidal silicon dioxide | 2.50 |
| Sodium lauryl sulfate | 1.00 |
| Color | 0.40 |
| Magnesium stearate | 1.05 |
| Talc | 0.25 |

Coating

| | | |
|---|---|---|
| Aquacoat ECD 30 solids (aqueous | 21.34 | Coated to a weight gain |
| ethyl cellulose dispersion) | | of about 12% by weight |
| Acryl eze white 9318509 | 11.75 | of the bilayered core |
| Dibutyl sebacate | 1.60 | |
| Triethyl citrate | 0.64 | |

Venlafaxine hydrochloride, HPMC, PVP K-30, lactose monohydrate and a part of Eudragit was mixed and granulated with purified water. The granules were dried, milled and lubricated with a mixture comprising magnesium stearate, talc and the remaining part of Eudragit.

Prosolv SMCC 90, colloidal silicon dioxide, crospovidone, sodium lauryl sulfate. Color, magnesium stearate and talc were mixed to obtain a blend. This was then compressed with the venlafaxine granules to obtain a bilayered core. The core was coated with a coating composition comprising ethyl cellulose and acryl eze to a weight gain of about 12% by weight of the core.

The tablets so obtained were subjected to dissolution testing using 900 ml of pH 6.8 phosphate buffer in United States Pharmacopoeia dissolution apparatus, type II, at a speed of 100 rpm. The results are recorded in Table 5 below,

TABLE 5

| Time (hours) | % venlafaxine released |
|---|---|
| 0 | 0 |
| 1 | 9 |
| 2 | 16 |
| 4 | 36 |
| 6 | 59 |
| 8 | 77 |
| 10 | 87 |
| 12 | 93 |
| 16 | 100 |

While the invention has been described by reference to specific embodiments, this was done for purposes of illustration only and should not be construed to limit the spirit or the scope of the invention.

1. An oral drug delivery system comprising:
a core comprising an active ingredient composition comprising therapeutically effective amount of at least one active ingredient and a pharmaceutically acceptable excipient and a composition selected from a swellable composition and a reactive composition located in an immediate vicinity of one or more preselected surfaces, and
a coating surrounding the core, said coating comprising a water-insoluble polymer and a pH-dependent polymer,
wherein the coating is reliably removed fully or partially from one or more of the preselected surfaces upon contact with intestinal fluids after a predetermined delay but not removed from at least one of the surfaces.

2. (canceled)

3. An oral drug delivery system as claimed in claim 1 wherein the coating on selected surface or surfaces of the tablet is selected from defective coatings and reactive coatings.

4. An oral drug delivery system as claimed in claim 1, wherein the active ingredient is an antidepressant agent.

5. An oral drug delivery system as claimed in claim 1, wherein the ratio of water-insoluble polymer to the pH-dependent polymer ranges from about 1:1 to about 10:1 by weight of the coating composition.

6. An oral drug delivery system as claimed in claim 5, wherein the system is coated to a weight gain of about 8% to about 15% by weight of the core.

7. An oral drug delivery system as claimed in claim 1, wherein the water-insoluble polymer is ethyl cellulose and the pH-dependent polymer is methacrylic acid copolymer.

8. An oral drug delivery system as claimed in claim 1, wherein the active ingredient composition is present as one or more layers and the swellable composition is present as one or more layers.

9. An oral drug delivery system as claimed in claim 8, wherein the active ingredient present in the different layers may be the same or different.

10. An oral drug delivery system as claimed in claim 1, wherein the active ingredient is a controlled release composition comprising therapeutically effective amount of an active ingredient and a release controlling excipient.

11. An oral drug delivery system as claimed in claim 10, wherein the release controlling excipient is selected from the group comprising high viscosity grades of hydroxypropyl methylcellulose (HPMC), polyethylene oxide homopolymers, hydroxypropyl cellulose (HPC) and mixtures thereof.

12. An oral drug delivery system as claimed in claim 11, wherein the release controlling excipient is used in an amount ranging from about 10% to about 50% by weight of the active ingredient composition.

13. An oral drug delivery system as claimed in claim 8, wherein the system further comprises a second active ingredient composition containing the same active ingredient as the active ingredient composition, wherein said second active ingredient composition is a controlled release composition, and wherein the active ingredient composition is a rapid releasing composition.

14. An oral drug delivery system as claimed in claim 1, wherein the swellable composition comprises a swelling agent.

15. An oral drug delivery system as claimed in claim 14, wherein the swelling agent is selected from the group comprising a swellable excipient, a gas generating agent and mixtures thereof.

16. An oral drug delivery system as claimed in claim 1, wherein the swellable composition comprises wicking agents.

17. An oral drug delivery system as claimed in claim 1, wherein the swellable composition comprises osmogents.

18. An oral drug delivery system comprising:
a core comprising an active ingredient composition comprising at least one active ingredient and a pharmaceutically acceptable excipient and a composition selected from a swellable and a reactive composition in form of an in-lay tablet located in an immediate vicinity of a preselected surface from which the coating is desired to be partially removed, and
a coating surrounding the core, said coating comprising a water-insoluble polymer and a pH-dependent polymer,
wherein the coating is reliably removed partially from the preselected surfaces upon contact with intestinal fluids after a predetermined delay.

19. An oral drug delivery system comprising:
a core composition comprising (i) an active ingredient composition comprising therapeutically effective amount of at least one active ingredient and pharmaceutically acceptable excipients, (ii) a swellable composition located in the immediate vicinity of one or more preselected surfaces, comprising a swelling agent selected from a swellable excipient, gas generating agents and mixtures thereof, and
a coating surrounding the core, said coating comprising a water-insoluble polymer and a pH-dependent polymer,
wherein the coating is impermeable to water and the active ingredient in gastric fluids, but is permeable to water in the intestinal fluids, whereby upon gastric emptying the system imbibes water and the swellable composition swells to rupture the coating on the preselected surface or surfaces.

20. An oral drug delivery system as claimed in claim 4, wherein the antidepressant agent is selected from the group comprising venlafaxine, paroxetine, fluvoxamine, sertraline, bupropion and their pharmaceutically acceptable salts.

21. The drug delivery system of claim 19 wherein the core comprises wicking agents.

22. The drug delivery system of claim 19 wherein the core comprises osmogents
